(19) United States
(12) Patent Application Publication
(10) Pub. No.: US 2018/0268927 A1
(43) Pub. Date: Sep. 20, 2018

(54) METHOD FOR CONSTRUCTING CLOUD INTEGRATION AND MANAGEMENT SYSTEM CAPABLE OF INTERACTIVELY OPERATING AND USING MEDICAL INFORMATION

(71) Applicants: Jean Fuh, New York, NY (US); Pei-Chin Chen, Hsinchu City (TW); Martin Mao Tsu Fuh, Taipei (TW)

(72) Inventors: Jean Fuh, New York, NY (US); Pei-Chin Chen, Hsinchu City (TW); Martin Mao Tsu Fuh, Taipei (TW)

(21) Appl. No.: 15/923,805

(22) Filed: Mar. 16, 2018

(30) Foreign Application Priority Data
Mar. 16, 2017 (TW) 106108781

(51) Int. Cl. G16H 10/60; G16H 50/20; H04L 29/08

(52) U.S. Cl. CPC G16H 10/60; H04L 67/10; G16H 50/20

(57) ABSTRACT

A method for constructing a cloud integration and management system capable of interactively operating and using medical information is disclosed. The method comprises creating or constructing a cloud-based medical information center architecture by using structural or nonstructural active files, where the medical information is collected from medical institutions. Within the system, the medical information is stored on an absolute time sequence using identifiable data of an individual. Health care information of an individual can be displayed on, gathered by, or transmitted by a personal home healthcare device or a wearable device. Such information can be imported to the cloud-based medical information center architecture.

### CROSS-REFERENCE TO RELATED APPLICATION(S)

This application claims the benefit under 35 U.S.C. § 119 of the earlier filing date of Taiwanese Patent Application No. 106108781, filed Mar. 16, 2017, which is incorporated by reference herein, in its entirety, and for all purposes.

### TECHNICAL FIELD

Embodiments of the present inventions relate to methods of a cloud integration and management system capable of interactively operating and using medical information, utilizing an absolute time sequence, is disclosed.

### BACKGROUND

So far, there is no cloud integration and management system that can successfully connect, interoperate, and apply long-term care information for acute and chronic diseases of an individual that is collected from a physiological information detection device authorized by governmental medical organizations, physiological information detected by an individual at home, and all medical information and data of diagnosis and treatment on acute and chronic diseases in domestic and foreign medical institutions approved by an individual in all health insurance systems.

### SUMMARY

Embodiments of the present invention relate to a method for constructing a cloud integration and management system capable of interactively operating and using medical information. Advancements in modern technology, especially in the fields of life and physiological information detection, computer, wireless communication, network, and cloud technologies, allow for the rapid commercialization of advanced physiological information detection devices and/or wireless communication devices. Such devices can be offered at a relatively inexpensive price that nearly everyone can have them. For example, most people have a smartphone for wireless communication that can make phone calls, send and receive an image, a photo, and a text message, and perform storage in a cloud network.

In addition, for marketing technologies and operation management in various industries, a computer and a cloud network can be used to generate analysis such as sales volumes by location, sales volumes by season, popularity of different products by gender and age, comparable products by different brands, and a comparison between quality and sales volumes. Despite extensive use and reliance on cloud technology, people are regrettably still unable to interactively operate and use all retrospective medical and lifestyle information (including information about clothing, food, housing, transportation, fertility, entertainment, sports, and the like) of a life of an individual with a time sequence on an absolute time sequence.

If such information and data are analyzed and processed against time by a cloud integration and management system, medical professionals in various fields can observe the occurrence and evolution processes of different particular diseases that the individuals are suffering from and the relationships among living environments, lifestyles, and social group cultures of the individuals, to facilitate disease prevention. One of the goals of the invention is to improve upon existing medical information management systems and to extend the scope of care from reactive to preventive.

Embodiments of the present invention introduces a new system of providing medical information by incorporating and tracking precise and holistic personal events, utilizing absolute time sequence as only one event may occur at a single point of time in an individual's life. The absolute time sequence tracking facilitates universal system integration as well as comprehensive and automatic data inter-operation.

This invention overcomes the present-day challenge of system integration of which data inter-operation is limited by geological, social, and economical barriers. Disease prevention through changes to living habits can be achieved through the tracking and integrating of long-term medical information an individual, understanding how diseases are formed and developed, performing medical diagnosis, and having the individual participate in various health management tasks.

The inventors of the instant application believe that advancements in computer technology, wireless communication and transmission technology, electronic encryption technology, network technology, cloud technology, and the like allow many organizations, institutions, companies, etc. to perform logistics, marketing, resource management and utilization to industries such as clothing, nutrition, housing, transportation, fertility, entertainment, and others. However, such advancements have not been utilized for a cloud integration and management system that can record and store all physiological information relating to disease treatment as well as lifestyle information (including information about clothing, food, housing, transportation, fertility, entertainment, sports, and the like in a social group culture) of the individual from prenatal stage until death. This information is organized by time and with structural or nonstructural active files. Structural files typically in a constant, fixed, or standardized format, and may be easily sorted, accessed and analyzed, such as numerical data. Nonstructural files are typically not in a constant, fixed, or standardized format, and may be difficult to sort, access, or analyze, such as text messages or surgical reports. The medical information and data of an individual is stored within a cloud integration and management system capable of performing interactive operations and applications, for analysis and processing, in order to prescribe medical treatment to the individual and further provide health management tasks throughout the life of the individual.

What is needed is a system capable of effectively and efficiently transmitting medical and lifestyle data and information of an individual and integrate the information to a cloud integration and management system by using structural or nonstructural active files, which may also be referred to as structural or nonstructural data, and analyze the data in detail to provide the scientific data for a medical professional of the individual to treat a disease and manage the health of the individual. Active files, as opposed to inactive files, require input of time at each data entry. Time is the only factor that all life forms have in common. The advancement of time cannot be reversed, and it is common to all types of life forms (including animals and plants). Time is fair, transparent, and the same and is irrespective of age, gender, race, or the like. An individual cannot go back in time or relive past experiences in a life span, and time is not affected by any human factor.

Conventionally, any organization, institution, or company that serves people through computer-based solutions generally involve operations that are performed or organized in a timeline. However, this timeline for such systems does not organize, correlate, nor analyze medical data in an absolute time sequence.

What is needed is a system where individuals can continuously transmit information from a device to a cloud integration and management system through the use of structural or nonstructural active files to create a specific and complete medical profile that is assigned to an individual. Further, the medical information of an individual, of the family of an individual, and or a greater group can be connected, stored, analyzed, and applied for generations. The inventors of the present invention describe herein a method for constructing a cloud integration and management system capable of interactively operating and using medical information.

One embodiment of the present invention relates to a method for a cloud integration and management system includes a first step of building a cloud-based medical information center system based on medical records from a comprehensive health insurance and allocating to the system a cloud integration function for comprehensive medical treatment and lifestyle information centered on an individual-by-individual basis.

A second step comprises generating personal identification information, including name, identity (ID) number, gender, date of birth, place of birth, address, mobile phone number, computer code, e-mail address, home medical equipment code or a wearable device code, skin color, hair color, race, and the like, and including the names of the biological parents (or guardians).

A third step comprises organizing the cloud-based medical information center system chronologically and, integrating, by the cloud-based medical information center system constructed in the first step, all medical service content by using structural or nonstructural active files in a track-type and dynamically stable time flowing and sorting program design, wherein an absolute time sequence comprises a time sequence based on year, month, date, hour, minute, and/or second, and the cloud-based medical information center system is directly connected to the time units of date, hour, minute, and second by using structural or nonstructural active files; and

A fourth step comprises correlating on an absolute time sequence for each individual, starting from prenatal stage or later up to death, medical diagnosis and treatment on the individual from medical institutions to a timeline that is a part of an absolute time sequence, transferring, transmitting or inputting in parallel lifestyle content and complete historical medical record information that are connected to the time sequence to said cloud-based medical information center system using structural or nonstructural active files, wherein, the active files for disease prevention and health management in the cloud-based medical information center system can be functionally integrated and used in conjunction with or applied to the individual's family, community, social networks, or other networks.

### BRIEF DESCRIPTION OF THE DRAWINGS

FIG. 1 is a diagram showing the transmission of medical information between a medical health insurance institution, a medical institution or a long-term care institution for chronic diseases, and an individual with medical health insurance.

FIG. 2 is a diagram of a method for creating a cloud integration and management system capable of interactively operating and using medical information according and embodiment of the present invention.

### DETAILED DESCRIPTION

Examples of cloud integration and management systems described herein may find use in technical fields such as computer technology, wireless communication and transmission technology, electronic password technology, network technology, cloud technology, and the like. Through the use of software and the foregoing technologies, personal health information and medical information can be collected starting from prenatal development. The medical information that is generated from birth to death that is distributed in institutions at home and abroad, personal long-term care information, and other physiological medical information and data (which may also include all information about clothing, food, housing, transportation, fertility, entertainment, social group cultures, lifestyle information, the Internet of things, and the like in the course of life of an individual) can be collected by using structural or nonstructural active files or data in a wide range and from multiple aspects. Based on a time sequence, a comprehensive analysis may be performed thoroughly to obtain a relationship among human health, medical diagnosis, and treatment until death. Embodiments of the present invention can perform such interactive operations and use health management information for the purpose of disease prevention for an individual.

Embodiments of the present invention relate to methods of constructing a cloud integration and management system capable of interactively operating and using medical information, where long-term care medical information for acute and chronic diseases can be obtained from a physiological information detection device, from physiological information and data detected by an individual at home, and from medical information of diagnosis and treatment in domestic and foreign medical institutions approved by an individual in all health insurance systems; information collected from various sources may be transmitted as input to a network of a cloud-based medical information system by using structural or nonstructural active files or structural or nonstructural data. All historical information and data about health, medical diagnosis and treatment, which can include the death of an individual, are recorded and stored in the cloud-based medical information system using an absolute time sequence as a main axis; and the information can be interactively operated and used for analysis and processing so as to provide medical diagnosis and treatment for an individual and provide personal health and life management for the individual by using scientific data and proofs.

Conventional systems may connect individual medical providers and its patients; and certain systems provide virtual clinics that allow patients to communicate with individual health care providers. However, conventional systems are limited with respect to the exchange of medical information. Even though individual medical providers utilize systems that record and analyze medical information such as medical history and medical record, the interactions are limited to the patient and individual medical providers. For example, if an individual has five different medical providers, then there are five unique sets of medical data. In this scenario, assuming that A1, A2, A3, A4, and A5 are 5 different medical providers and B is the patient, then there are 5 different sets of medical data exchange happening between A1 and B, A2 and B, A3 and B, A4 and B and A5 and B. Although patient B can request that provider A1 share information with provider A2 but the information exchange would not be persistent or interoperable. Moreover, even though there are clinics and hospitals that are within a single network, medical information generated within the network merely represents a bigger version of a single medical provider. The present invention seeks to provide a solution to this deficiency.

Embodiments of the present invention relate to methods for creating or constructing a cloud integration and management system capable of interactively operating and using medical information. The rapid development of technology, especially in the industries and fields of computers, servers, and cloud technologies, has led to a reduction of hardware size, rapid storage and transmission of a large quantity of data, and inexpensive hardware. For example, nearly everyone has a smartphone to make a call, and transmit many favorite pictures, photos, and text messages to a cloud network for storage.

Likewise, medical technology has also advanced—such as micro-message detection technology, which allows medical personnel to provide diagnosis and treat during an initial stage of a disease. Because many diseases are progressive, early detection and disease prevention of disease is possible by monitoring and analyzing the gradual changes of various physiological information continually detected by a human and a medical history. An important concept is that disease prevention is more important than medical treatment. Disease prevention can greatly reduce health care costs and can prevent pain and suffering. Embodiments of the present invention relate to providing personalized medical treatment to an individual and to further provide health management throughout the life of the individual by collecting and analyzing scientific data.

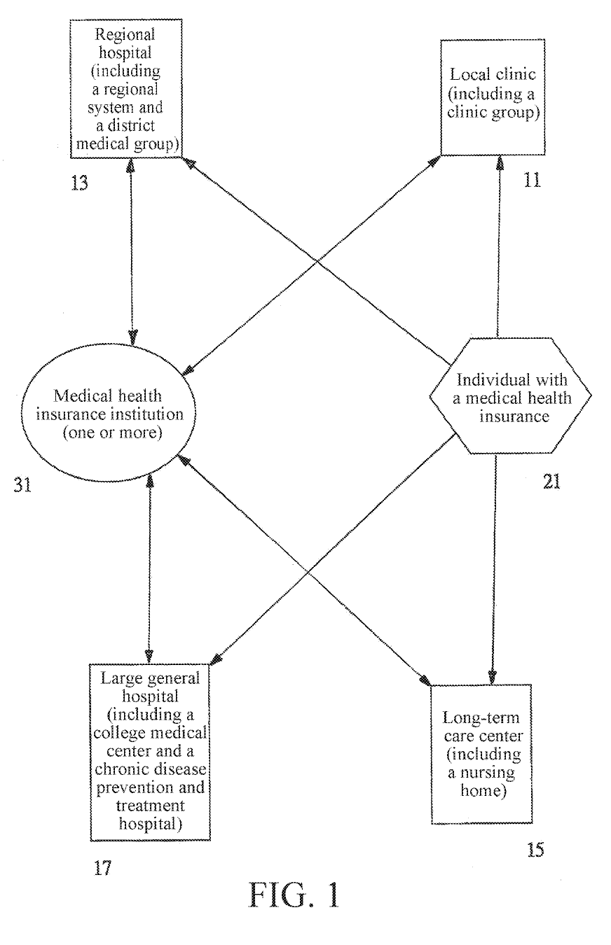

Referring to FIG. 1, FIG. 1 is a diagram of the relationship showing the transmission of medical information between a medical health insurance institution, a medical institution or a long-term care institution for chronic diseases, and an individual with medical health insurance. In the figure, transmissions of medical information are represented by a solid line with a single-headed arrow and a solid line with double-headed arrows. A solid line with a single-headed arrow means that the medical information is uni-directionally transmitted to an arrow direction. A solid line with double-headed arrows displays that the medical information is bi-directionally transmitted to arrow directions. In the figure, medical information is not transmitted between a medical health insurance institution 31 (an insurance institution is represented by a circle) and an individual 21 with medical health insurance (the insured is represented by a hexagon); therefore, they are not connected by using a solid line with a single-headed arrow or a solid line with double-headed arrows. However, after the individual 21 with medical health insurance pays health insurance fees to the medical health insurance institution 31 or the state pays the health insurance fee whenever the individual 21 with medical health insurance requires medical care, the individual 21 may seek medical advice in a medical institution (a medical institution is represented by a rectangle) such as a local clinic 11, a regional hospital 13, a long-term care center 15, or a large general hospital 17. In this case, the individual 21 with medical health insurance creates medical information of a pain or a disease in the body and provides the medical information to the medical institution such as the local clinic 11, the regional hospital 13, the long-term care center 15, or the large general hospital 17 for diagnosis and treatment. Therefore, the individual 21 with medical health may transmit or generate medical information to various institutions, as represented through the solid lines with a single-headed arrow, and where single-headed arrow points to each medical institution represented by a rectangle. After the individual 21 with a medical health insurance is diagnosed and treated in each medical institution such as the local clinic 11, the regional hospital 13, the long-term care center 15, or the large general hospital 17, medical information related to the diagnosis and treatment on the individual 21 with medical health insurance and medical information related to insurance payments is transmitted to the medical health insurance institution 31. Then, the medical health insurance institution 31 transmits the medical information related to insurance payments and a medical health service project thereof or changed content to the local clinic 11, the regional hospital 13, the long-term care center 15, or the large general hospital 17. Therefore, there is a connection where medical information is exchanged between the medical health insurance institution 31 and each medical institution such as the local clinic 11, the regional hospital 13, the long-term care center 15, or the large general hospital 17 is represented by a solid line with double-headed arrows, and arrow directions point to two parties to indicate mutual medical information transmission. It can be observed from FIG. 1 that there is a connection relationship represented by a solid line with a single-headed arrow or a solid line with double-headed arrows that, the individual 21 with medical health insurance will seek medical advice from various medical institutions in different places when medical care is required. However, whenever the individual 21 with medical health insurance needs to obtain past medical information generated from prior visits to the local clinic 11, the regional hospital 13, the long-term care center 15, or the large general hospital 17, the individual 21 with medical health insurance needs to return to each respective medical institution in which the individual 21 with medical health insurance has sought medical advice each time to obtain such medical information (such as a diagnosis and treatment report), because each of current medical institutions such as the local clinic 11, the regional hospital 13, or the long-term care center 15, or the large general hospital 17 separately stores or manages the medical information of the individual 21 with medical health insurance. Consequently, currently, in such a closed medical information processing system, medical information cannot be exchanged or interactively operated and used, which may lead to a waste of unnecessary medical resources and cannot improve quality of a medical service.

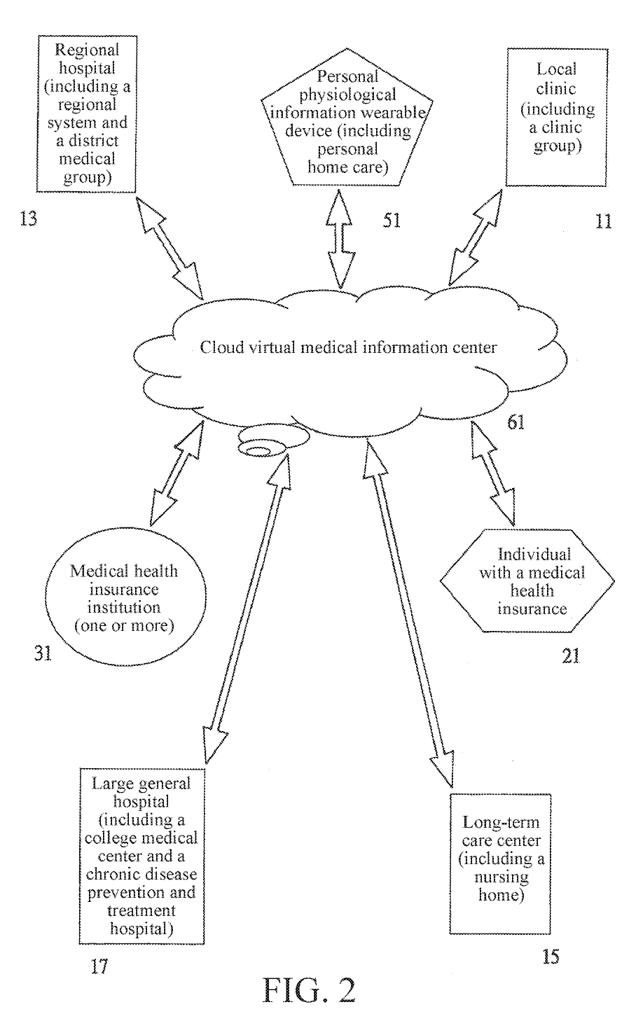

Referring to FIG. 2, FIG. 2 is a diagram of a method for creating or constructing a cloud integration and management system capable of interactively operating and using medical information according to an embodiment of the present invention. The figure is different from current medical systems. Embodiments of the present invention provide a platform to collect and restructure medical information on an absolute time sequence to achieve data interoperability between medical providers and patients. Data that can be organized or cross-referenced on an absolute time sequence include patient data, non-patient data, various life activities and events. The data from lifestyle, culture, and environment may be used to generate preventive care recommendations. Improved data interoperability leads to increased coverage, transparency, and quality in health care. Moreover, the platform allows for the holistic collection of data.

FIG. 2 displays a relationship of transmission and integration and management of medical information between a personal physiological information wearable device 51 (including personal home care, and represented by a pentagon), a cloud-based (cloud virtual) medical information center 61 (represented by a cloud shape), a medical institution (represented by a rectangle) such as a local clinic 11, a regional hospital 13, a long-term care center 15, or a large general hospital 17, a medical health insurance institution 31 (represented by a circle), an individual 21 with medical health insurance (represented by a hexagon). In FIG. 2, hollowed double-headed arrows point to the cloud-based medical information center 61 to indicate connections to the cloud-based medical information center 61, representing that the medical information is transmitted bi-directionally. More importantly, the cloud-based medical information center 61 collects and integrates all information in the personal physiological information wearable device 51 (represented by a pentagon) of each individual, the medical institution (represented by a rectangle) such as the local clinic 11, the regional hospital 13, the long-term care center 15, or the large general hospital 17, the medical health insurance institution 31 (represented by a circle) and the individual 21 with medical health insurance (represented by a hexagon). Medical information generated from past to currently is continually transferred and input, in parallel, to the cloud-based medical information center by using structural or nonstructural active files or structural or nonstructural data. Upon receipt of medical information, the cloud-based medical information center 61 immediately operates, analyzes, and processes medical information and data of each individual. After the various medical entities have been authenticated (including the cloud-based medical information center 61), medical information such as disease diagnosis and treatment information of each individual 21 with medical health insurance can be obtained from the cloud-based medical information center 61 at any time and any place upon connection to the cloud-based medical information center 61 from the Internet. For example, when the individual 21 with medical health insurance has a disease and seeks medical advice in the large general hospital 17, all past detection, diagnosis, and treatment reports of the individual 21 with medical health insurance in different medical institutions such as the local clinic 11, the regional hospital 13, the long-term care center 15, or the large general hospital 17 at different places can be obtained from the cloud-based medical information center 61 after authentication with an ID number and identification data of the individual 21 with medical health insurance succeeds. In addition, daily data (the data includes personal home care information) of the individual 21 with medical health insurance in the personal physiological information wearable device 51 can be obtained. All medical information of an individual 21 with medical health insurance is sequentially arranged in an absolute time sequence. A medical professional may clearly see, from a computer screen, past and present physical conditions of the individual 21 with medical health insurance so as to more accurately diagnosis and provide treatment on the individual 21 with medical health insurance. Further, as shown in FIG. 2, the cloud-based medical information center 61, in accordance with an embodiment the present invention, may be connected to the personal physiological information wearable device 51 (including personal home care information, and represented by a pentagon), the medical health insurance institution 31 (represented by a circle), the medical institution (represented by a rectangle) such as the local clinic 11, the regional hospital 13, the long-term care center 15, or the large general hospital 17, and the individual 21 with medical health insurance (represented by a hexagon) to collect medical information. The cloud-based medical information center 61 can arrange all the medical information on an absolute time sequence, so that medical information that is generated in real time can be observed from the computer screen, and retrospective medical information and data can be operated, analyzed, and processed.

In addition, when the individual 21 with medical health insurance can participate in their own health management, and where medical information between individuals or between an individual and a social group is continually integration, the quality of medical service can be improved. According to an embodiment of the present invention, personal medical information from medical health insurance institution 31 can be distributed from medical institutions to various places. In addition, real-time physiological information detected by personal physiological information wearable device 51 of the individual 21 with medical health insurance, and information in the long-term care center 15 (including a nursing home) can be integrated into and distributed from the cloud-based medical information center 61. All medical information associated with an individual 21 with medical health insurance is integrated on an absolute time sequence using structural or nonstructural active files or structural or nonstructural data. From various physiological information gather from an individual and a group, by means of evaluation by multi-level professionals and processes such as statistics collection, analysis, summarization, and continual repeated verification, improved medical diagnosis and treatment can be achieved for the individual 21 with medical health insurance. Moreover, this information can be functionally connected and integrated to a family group or other forms of networks for disease prevention.

### REFERENCE NUMERALS

11 Local clinic (including a clinic group)
13 Regional hospital (including a regional system and a district medical group)
15 Long-term care center (including a nursing home)
17 Large general hospital (including a college medical center and a chronic disease prevention and treatment hospital)
21 Individual with a medical health insurance
31 Medical health insurance institution
51 Personal physiological information wearable device (including personal home care)
61 Cloud-based (cloud virtual) medical information center

What is claimed is:

1. A method comprising:
creating a cloud-based medical information center system based on medical records from multiple institutions and allocating to the system a cloud integration function for comprehensive medical treatment and lifestyle information centered on an individual;
gathering or storing personal identification information of the individual, comprising name, identity number, gender, date of birth, place of birth, address, mobile phone number, computer code, e-mail address, home medical equipment code or wearable device code, skin color, hair color, race, and including the names of biological parents or guardians;
organizing the cloud-based medical information center system along an absolute time sequence and integrating medical information through the use of structural or nonstructural active files within said cloud-based medical information center system, where the absolute time sequence can be based on year, month, date, hour, minute, or second, and said cloud-based medical information center system is directly connected to time units of date, hour, minute, and second by using structural or nonstructural active files; and
correlating medical diagnosis and treatment information from medical institutions on an absolute time sequence, then transferring, transmitting or inputting in lifestyle content and complete historical medical record information on the absolute time sequence to said cloud-based medical information center system using structural or nonstructural active files, wherein the active files for disease prevention and health management in said cloud-based medical information center system can be functionally integrated and used in conjunction with or applied to data from other individuals or networks.
